(19) United States
(12) Patent Application Publication
(10) Pub. No.: US 2008/0125584 A1
(43) Pub. Date: May 29, 2008

(54) Salts Assisted Selective Extraction Of 6-Acetyl- 4,1' , 6' Trichlorogalactosucrose And 4,1', 6' Trichlorogalactosucrosse From Aqueous Reaction Mixture

(76) Inventors: Rakesh Ratnam, Maharashtra (IN); Sundeep Aurora, Maharashtra (IN)

Correspondence Address: NATH & ASSOCIATES, 112 South West Street, Alexandria, VA, 22314, US

(21) Appl. No.: 11/792,620

(22) PCT Filed: Dec. 9, 2005
(86) PCT No.: PCT/IN05/00408

§ 371 Date: Jun. 8, 2007

(30) Foreign Application Priority Data
Dec. 10, 2004 (IN) 1316/MUM/2004

(51) Int. Cl. C07H 1/06

(52) U.S. Cl. 536/127

(57) ABSTRACT

A process is described to increase efficiency of extraction of 4,1′,6′ trichlorogalactosucrose (TGS) from aqueous solution by addition of salt. The invention helps in reducing the quantity of solvent needed for achieving substantial extraction. The invention also leads to extraction of TGS substantially free from polar impurities.

### TECHNICAL FIELD

The present invention relates to a process and a novel strategy for synthesis of chlorinated sucrose, 1′-6′-Dichloro-1′-6′-DIDEOXY-β-Fructofuranasyl-4-chloro-4-deoxy-galactopyranoside.

### BACKGROUND OF THE INVENTION

Chlorinated sucrose preparation is a challenging process due to the need of chlorination in selective less reactive positions in sucrose molecule in competition with more reactive positions. Generally, this objective is achieved by a procedure which involves essentially protecting the primary hydroxy group in the pyranose ring of sugar molecule by converting it to either aromatic or aliphatic esters or orthoesters, and the protected sucrose is then chlorinated in the desired positions (1′, 6′ &, 4) to give the acyl or aryl ester derivative of the product, which is then deesterified to give the desired product 1′-6′-Dichloro-1′-6′-DIDEOXY-β-Fructofuranasyl-4-chloro-4-deoxy-galactopyranoside i.e. 4,1′,6′ trichlorogalactosucrose (TGS).

Strategies of prior art methods of production of TGS are based on following: Sucrose-6-acetate is chlorinated by Vilsmeier-Haack reagent to form 6-acetyl-4,1′,6′trichlorogalactosucrose (6-acetyl-TGS). After chlorination, the deacetylation of 6-acetyl-TGS to TGS is carried out in the reaction mixture itself. The TGS is then purified from the reaction mixture in various ways based on selective extraction into water immiscible solvent or solvents.

Thus, in the existing strategy, the selective extraction of TGS into water immiscible solvent or solvents that have low miscibility in water is required to be done in the last stages of the process. This step is beset with a few following problems.

The solubility of TGS is very high in aqueous solutions, with the result that while extracting from aqueous solutions into solvent of low miscibility in water, large volumes of solvents and repetitive extractions of TGS are required. This process is therefore less efficient and time consuming.

Further, it was also found that when the aqueous solution of reaction mixture containing TGS is extracted with organic solvents to ensure satisfactory extent of extraction of the product TGS, the product TGS recovered from such extract invariably contained a polar impurity which traveled very close to TGS in a TLC assay (FIG. 1). This impurity is difficult to be avoided in this way of extraction of the product TGS.

It was an objective of the present invention to find out more efficient method for extraction of TGS from aqueous solvent to water immiscible or nearly immiscible organic solvents. It was also an object of the present invention to extract the product TGS free from polar impurity.

### SUMMARY OF INVENTION

In present invention, it was found that TGS in aqueous solution could be selectively extracted into organic solvents which are water immiscible or nearly immiscible solvents in relatively lesser quantity of solvent, if the aqueous solution containing TGS is saturated with salts, including but not limited to sodium chloride, sodium acetate, Calcium Chloride, Barium Chloride, Potassium Chloride, Potassium Acetate, etc. It was also found that the organic solvent extract obtained after extraction of salt saturated aqueous solution of the TGS contained very little of the polar impurities. Thus, this method not only extracted the TGS totally into the organic solvent but it also was a method for removal of polar impurities from the crude TGS.

It was seen that improvement of extraction from aqueous solutions to nearly water immiscible or water immiscible organic solvent on account of salt saturation was seen to be true even for 6-acetyl-TGS.

This invention covers extraction of TGS as well as 6-acetyl-TGS from any process stream in process of manufacture of TGS, i.e. aqueous or non-aqueous solution derived in the process of manufacture of 6-acetyl-TGS or TGS including and in addition to the aqueous reaction mixture derived from deacylation reaction applied to pure solution containing 6-acetyl-TGS, or to a reaction mixture containing TGS. This enlisting of 6-acetyl-TGS &/or TGS solutions to which this invention covers within its scope is not claimed to be exhaustive and any analogous 6-acetyl-TGS &/or TGS solution which is subjected to the process described in this invention for the purpose of 6-acetyl-TGS &/or TGS extraction is also covered within the scope of this specification.

### BRIEF DESCRIPTION OF FIGURES

FIG. 1: Thin Layer Chromatography profile of product TGS before and after extraction from salt saturated solution by organic solvents

### DETAILS OF INVENTION

Reaction mixtures containing 6-acetyl-TGS &/or TGS which this invention shall cover as subject matter within its scope are encountered in many different ways. It may be as simple as derived by dissolution of 6-acetyl-TGS &/or TGS required to be purified further and dissolved in water for further purification processing. It is also usually encountered in the form of reaction mixtures which are already aqueous or can be made aqueous by driving out the organic solvents to required degree, and end products of unit processes such as column chromatography, solvent extractive purification, crystallization followed by their dissolution in water, precipitation followed by their dissolution in water, drying of a solution containing 6-acetyl-TGS &/or TGS with or without other chemicals and its dissolution in water in methods described in several prior art patents and patent applications including processes described by Mufti et al. (1983) U.S. Pat. No. 4,380,476, Walkup et al. (1990 No. 4980463), Jenner et al. (1982) U.S. Pat. No. 4,362,869, Tulley et al. (1989) U.S. Pat. No. 4,801,700, Rathbone et al. (1989) U.S. Pat. No. 4,826,962, Bornemann et al (1992) U.S. Pat. No. 5,141,860, Navia et al. (1996) U.S. Pat. No. 5,498,709, Simpson (1989) U.S. Pat. No. 4,889,928, Navia (1990) U.S. Pat. No. 4,950,746, Neiditch et al. (1991) U.S. Pat. No. 5,023,329, Walkup et al. (1992) U.S. Pat. No. 5,089,608, Dordick et al. (1992) U.S. Pat. No. 5,128,248, Khan et al. (1995) U.S. Pat. No. 5,440,026, Palmer et al. (1995) U.S. Pat. No. 5,445,951, Sankey et al. (1995) U.S. Pat. No. 5,449,772, Sankey et al. (1995) U.S. Pat. No. 5,470,969, Navia et al. (1996) U.S. Pat. No. 5,498,709, Navia et al. (1996) U.S. Pat. No. 5,530,106 and patent applications containing similar patentable matter including in co-pending application Nos. WO 2005/090374 A1 and WO 2005/090376 A1, wherein, in the process of production, 6-acetyl-TGS is produced and TGS is produced in the reaction mixture as a result of deacetylation of chlorination reaction mixture containing 6-acetyl-TGS. This list is only illustrative and not exhaustive and any 6-acetyl-TGS &/or TGS containing aqueous solution in a process of production of 6-acetyl-TGS &/or TGS and related products are included within the scope as embodiments of a process of extraction of TGS from a TGS containing solution to which this invention covers.

One such process illustrating formation of 6-acetyl-TGS &/or TGS in aqueous reaction mixture starts with chlorination of sucrose-6-acetate with Vilsmeier Haack reagent. The reaction mass is cooled to room temperature and centrifuged to remove suspended solids. The filtrate is passed through Agitated Thin Film Dryer (ATFD), to remove dimethylformamide (DMF). Details on ATFD are as per given in above referred patent applications WO 2005/090374 A1 and WO 2005/090376 A1. The solid mass obtained after ATFD becomes free from DMF, which is confirmed by GC analysis.

The ATFD solids which contain 6-acetyl-TGS and other inorganic salts, are dissolved in 3-8 times or more preferably 3-4 times w/v of water. The pH is to be adjusted to neutral and suspended solid in the aqueous solution removed by filtration. The presence of 6-acetyl-TGS in the solution is analyzed by Thin Layer Chromatography (TLC) and High Pressure Liquid Chromatography (HPLC). Either this 6-acetyl-TGS &/or TGS formed after deacetylation can be extracted from this aqueous solution by solvents including ethyl acetate, butyl acetate, any alkyl ester solvent, MTBE etc.

It was found in present invention that the problem of requirement of repeated extraction with large volume of solvent for satisfactory extraction could be overcome surprisingly by a very simple method of addition of salt to aqueous solution prior to extraction with water immiscible or sparingly miscible solvents such as ethyl acetate, butyl acetate, methyl ethyl ketone, etc., which further enables the reduction of solvent consumption when compared to extraction without salt saturation for achieving comparable degree of almost complete extraction. This benefit was more spectacular for TGS than for 6-acetyl-TGS and examples related to TGS are given in more details below to illustrate the invention.

Further, it has also been found that the solvent extraction of a salt saturated aqueous solution of TGS helps in leaving behind polar impurities in aqueous solution.

This invention describes a method that is applicable to any aqueous solution of 6-acetyl-TGS &/or TGS, preferably substantially free from DMF, from which it is necessary to make its extraction/isolation of 6-acetyl-TGS &/or from that solution for any purpose.

The ranges of reaction conditions given in this specification and in the example given below are for the purpose of illustrating the working of this invention and are not meant to limit the scope of the invention and any variation in the same which is reasonable and obvious to the person skilled in the art is covered within the scope of this specification. The scope of this specification includes analogous reactants and reactions of analogous generic nature.

Anything mentioned in singular is also construed to include plural as per the context viz. a mention of “a method of producing 6-acetyl-TGA” covers all methods of producing 6-acetyl-TGA.

### EXAMPLE 1

Aqueous layer, 80 L, containing 15 kg TGS with and without saturation with salt was extracted with various organic solvents. Results obtained are given below in Table no. 1

TABLE 1

| Ethyl | | | | | |
|---|---|---|---|---|---|
| acetate for | | Methyl | | Methyl | |
| complete extraction | | ethyl ketone for | | Isobutyl ketone | |
| of TGS | | complete extraction of | | for complete extraction | |
| From | | TGS | | of TGS | |
| Salt | Without | From Salt | Without | From Salt | |
| saturated | salt | saturated | salt | saturated | Without salt |
| aqueous | saturation | aqueous | saturation | aqueous | saturation |
| 280 L | 480 L | 80 L | 240 L | 120 L | 360 L |

Thus, it is clear that salt saturation of aqueous solution before extraction of TGS by water immiscible or nearly immiscible organic solvents reduces significantly the solvent required for practically complete extraction of TGS.

### EXAMPLE 2

65 kg of 6-O-acetyl sucrose was taken for the chlorination reaction. The chlorination was carried out using the Vilsmeier Haack reagent generated from PCl₅ and DMF. 1000 L of chlorinated reaction mass was neutralized to pH 5-6.

The reaction mass was cooled to room temperature (25-30° C.) and centrifuged to remove suspended solids. The filtrate was passed through Agitated Thin Film Dryer (ATFD), to remove DMF. Details on ATFD are as per given in the patent applications WO2005/090374 A1 and WO2005/090376 A1. The solids obtained after ATFD were tested for DMF absence by gas chromatographic (GC) analysis.

The ATFD solids (400 kg) which contain 6-acetyl-TGS and other inorganic salts, were dissolved in 3-4 times w/v of water. The same could have been dissolved in any other volume range between 3 to 8. The pH was adjusted to 9.0-9.5 using calcium hydroxide slurry and deacetylation was monitored by TLC. After the deacetylation, the pH of the deacetylated mass was adjusted to neutral and filtered using appropriate filter aid to remove suspended solids.

The DMF free aqueous solution was saturated with salt and then extracted with ethyl acetate. The quantity of solvent required to achieve practically complete extraction of TGS from aqueous solution with and without salt saturation in various solvents is given below. The TGS content in the pooled extracts at the end as determined by HPLC was found to be 30 kg. The salt saturation carried out in the aqueous medium is at 100% saturation level.

TABLE 2
Volume of solvents to achieve practically complete extraction of
30 kg TGS in organic solvents with and without salt saturation

| Ethyl acetate for | | Methyl | | | |
|---|---|---|---|---|---|
| complete extraction | | ethyl ketone for | | Methyl Isobutyl | |
| of TGS | | complete extraction of | | ketone for complete | |
| From | | TGS | | extraction of TGS | |
| Salt | Without | From Salt | Without | From Salt | Without |
| saturated | salt | saturated | salt | saturated | salt |
| aqueous | saturation | aqueous | saturation | aqueous | saturation |
| 3200 L | 7000 L | 1600 L | 4000 L | 1600 L | 4500 L |

The ethyl acetate extraction with various levels of salt saturation also was studied for TGS extraction. Qty of TGS taken for extraction was 30 kg in 1600 L of aqueous solution. Qty. of ethyl acetate taken for extraction was 1:1 of the aqueous solution for extraction. The figures of TGS in kg pertain to TGS extracted in the first round of extraction with ethyl acetate in the conditions described above. Results are given in Table 3 below.

TABLE 3

| Level of Salt | |
|---|---|
| saturation | TGS extracted in kg |
| 0% | 6.85 kg |
| 20% | 6.96 kg |
| 40% | 7.65 kg |
| 60% | 8.65 kg |
| 80% | 10.8 kg |
| 100% | 14.5 kg |

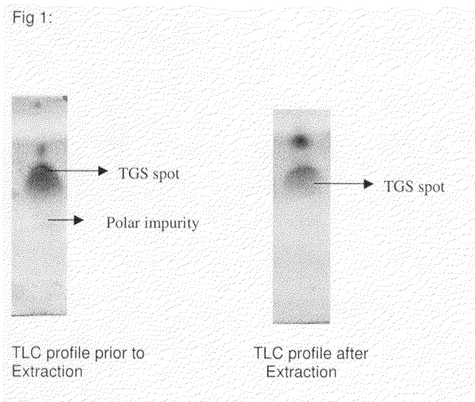

The reaction mixture contained polar impurity besides TGS which traveled very close to TGS in TLC assay as shown in FIG. 1. These are usually the polar impurities which are difficult to remove completely by column chromatography. When this reaction mixture was saturated and then extracted with organic solvent, the product TGS after TLC assay was seen to be practically free from this polar impurities.

The polar impurity profile in the extracted ethyl acetate from the above experiment is given in Table 4 as follows:

TABLE 4

| | | Qty. of TGS extracted |
|---|---|---|
| | | at various levels of |
| | Polar impurities | salt saturation |
| Level of Salt | (Dichloro derivatives | (already shown in |
| saturation | of sucrose | Table: 3 |
| 0% | 0.058 kg | 6.85 kg |
| 20% | 0.055 kg | 6.96 kg |
| 40% | 0.052 kg | 7.65 kg |
| 60% | 0.043 kg | 8.65 kg |
| 80% | 0.021 kg | 10.8 kg |
| 100% | 0.014 kg | 14.5 kg |

1. A process of extraction of 6-acetyl-4,1′,6′ trichlorogalactosucrose (6-acetyl-TGS) and/or 4,1′,6′ trichlorogalactosucrose, (TGS), from aqueous solution, containing or not containing other solutes or suspended particles, by an organic solvent including one or more of ethyl acetate, butyl acetate, methyl tertiary butyl ether (MTBE), other ketonic solvents, wherein:
a) the said aqueous solution is substantially free from organic solvents including a tertiary amide including dimethylformamide,
b) one or more salt/s is/are added to the aqueous solution prior to the extraction by organic solvent
c) the added salt or salts is/are in a concentration enough to effect improved solubility of 6-acetyl-TGS and/or TGS in the extracting solvent, and
d) optionally, the added salt or salts is/are in a concentration enough to effect selective extraction of 6-acetyl-TGS and/or TGS substantially free from polar impurities.

2. A process of claim 1 wherein the said salt or salts include one or more of sodium chloride, calcium chloride, barium chloride or potassium chloride.

3. A process of claim 2 wherein the said concentration of salt added is preferably in a quantity enough to reach saturation or near saturation.

4. A process of claim 3 wherein the said aqueous solution includes one or more of following:
a) TGS dissolved in water for the purpose of further purification,
b) a process stream from a process of production of TGS
